(19) United States
(12) Patent Application Publication
(10) Pub. No.: US 2014/0349351 A1
(43) Pub. Date: Nov. 27, 2014

(54) Production of 5-Hydroxymethylfurfural From Fructose

(71) Applicant: Novozymes A/S, Bagsvaerd (DK)

(72) Inventors: Jacob Skibsted Jensen, Herlev (DK); Thomas Grotkjaer, Frederiksberg (DK); Sven Pedersen, Gentofte (DK); Rolf Ringborg, Herlev (DK)

(73) Assignee: Novozymes A/S, Bagsvaerd (DK)

(21) Appl. No.: 14/351,108

(22) PCT Filed: Oct. 11, 2012
(86) PCT No.: PCT/EP2012/070151

§ 371 Date: Apr. 10, 2014

Related U.S. Application Data
(60) Provisional application No. 61/548,831, filed on Oct. 19, 2011.
(60) Provisional application No. 61/640,958, filed on May 1, 2012.

(30) Foreign Application Priority Data
Oct. 12, 2011 (EP) 11184895.8

(51) Int. Cl. C07D 307/50; C12P 17/04

(52) U.S. Cl. CPC C07D 307/50; C12P 17/04
USPC 435/126; 549/488

(57) ABSTRACT

The present invention relates to a process for producing 5-hydroxymethylfurfural (HMF) from fructose in a single-phase aqueous solution comprising an organic solvent.

### FIELD OF THE INVENTION

The present invention relates to a process for producing 5-hydroxymethylfurfural (HMF) from fructose in a single-phase aqueous solution comprising an organic solvent.

### BACKGROUND OF THE INVENTION

Many chemical compounds needed for various industries have for many years been derived from the petrochemical industry. However, due to increases in the price of crude oil and a general awareness of replacing petrochemicals with renewable resources there has been and still is a wish to base the production of chemical compounds on renewable resources.

5-hydroxymethylfurfural is an example of such a compound because it is derived from dehydration of sugars making it derivable from renewable biomass resources. HMF can for example be converted to 2,5-dimethylfuran by hydrogenolysis of C—O bonds over a copper-ruthenium (CuRu) catalyst (Roman-Leshkov Y et al., Nature, 2007, 447 (7147), 982-U5), which is a liquid biofuel or to 2,5-furandicarboxylic acid by oxidation (Boisen A et al., Chemical Engineering Research and Design, 2009, 87(9), 1318-1327). The latter compound, 2,5-furandicarboxylic acid, can be used as a replacement of terephthalic acid in the production of polyesters such as polyethyleneterephthalate (PET) and polybutyleneterephthalate (PBT).

US 2008/0033188 discloses a catalytic process for converting sugars to furan derivatives, e.g. 5-hydroxymethylfurfural, using a biphasic reactor containing a reactive aqueous phase and an organic extracting phase.

US 2009/0030215 discloses a method of producing HMF by mixing or agitating an aqueous solution of fructose and inorganic acid catalyst with a water immiscible organic solvent to form an emulsion of the aqueous and organic phases.

U.S. Pat. No. 7,317,116 discloses a method for utilizing an industrially convenient fructose source in a dehydration reaction, converting a carbohydrate to a furan derivative.

Huang R et al., 2010, Chem. Comm., 46, 1115-1117 discloses the integration of enzymatic and acid catalysis for the selective conversion of glucose into HMF, where borate-assisted isomerase was used to convert glucose into fructose and the resulting sugar mixtures were then dehydrated in water-butanol media to produce HMF.

Bicker M et al., 2003, Green Chem, 2003, 5, 280-284, studied the dehydration of D-fructose (10 g L⁻¹) in an acetone-water mixture (90:10) with sulfuric acid as catalyst by varying the parameters of temperature, pressure, catalyst concentration, solvent composition and residence time, as well as the influence of water content in a mixture varied from 10 Vol.-% water in acetone to pure water where the sulfuric acid concentration, the temperature and the pressure were kept constant.

In the industrial manufacture of high-fructose corn syrup, glucose is often converted into fructose by a process catalyzed by the enzyme xylose isomerase (E.C. 5.3.1.5) which for these reasons is usually called a “glucose isomerase”.

Glucose can be isomerized to fructose in a reversible reaction. Under industrial conditions, the equilibrium is close to 50% fructose. To avoid excessive reaction times, the conversion is normally stopped at a yield of about 45% fructose.

Glucose isomerase (GI) is one of the relatively few enzymes that are used industrially in an immobilized form. One reason for immobilization is to minimize the reaction time in order to prevent degradation of fructose to organic acids and carbonyl compounds that inactivate the enzyme. The substrate to the GI-columns is highly purified to avoid clogging of the bed and destabilization of the enzyme. The recommended conductivity is <50 μS/cm.

The most commonly used commercially available immobilized glucose isomerases are SWEETZYME™ IT (Novozymes A/S, Denmark), an enzyme from S. murinus crosslinked with glutaraldehyde; GENSWEET™ (Genencor Int. Inc, US), an enzyme from S. rubigonosus crosslinked with or without cellular debris using polyethylene imine and glutaraldehyde; and AGI-S-600™ (Godo Shusei, Japan), an enzyme from S. griseofuseus treated with chitosan and glutaraldehyde. Other ways of producing fructose is by hydrolysis of sucrose to obtain a composition comprising glucose and fructose in a 50:50 ratio or by catalytic conversion of mannose with mannose isomerase to fructose.

### SUMMARY OF THE INVENTION

In a first aspect, the invention provides a process for producing 5-hydroxymethylfurfural, said process comprising:
a) providing an aqueous solution comprising fructose and, optionally, glucose and/or mannose;
b) optionally contacting the solution with glucose isomerase enzyme (E.C. 5.3.1.5) which converts glucose to fructose and/or mannose isomerase enzyme (E.C. 5.3.1.7) which converts mannose to fructose;
c) combining the solution with at least one organic solvent as well as an acid catalyst and/or a salt to provide a reaction mixture, wherein the mixture forms a single-phase system at standard conditions of 20° C. and 1 atm. absolute pressure; and
d) heating said reaction mixture for a time sufficient to allow dehydration of fructose to provide 5-hydroxymethylfurfural in a resulting product mixture.

### BRIEF DESCRIPTION OF DRAWINGS

FIG. 1 shows a process diagram of a HMF-production process according to the invention.

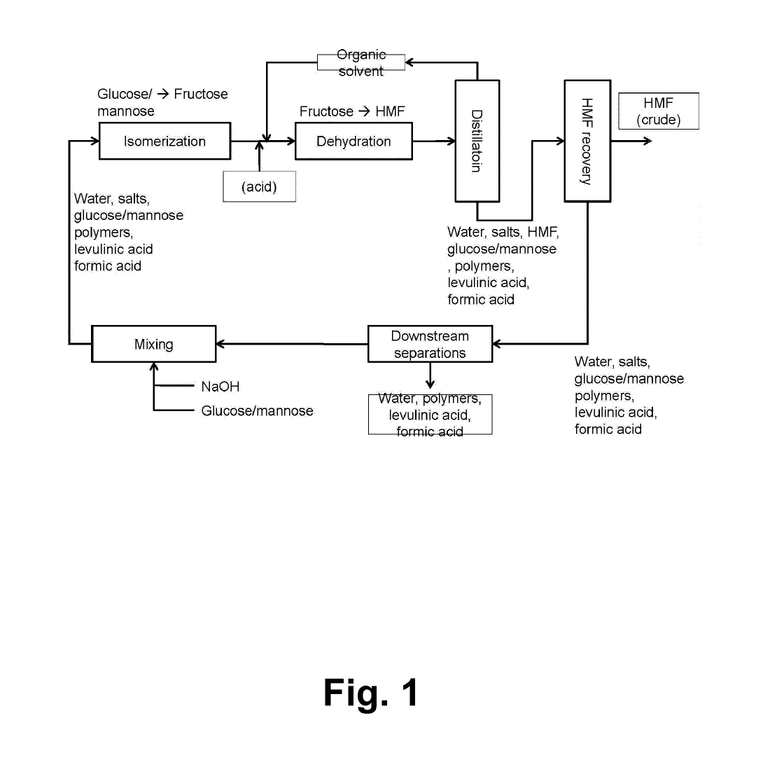

FIG. 2 shows a process diagram comprising a preheater unit.

### DEFINITIONS AND ABBREVIATIONS

The terms “5-hydroxymethylfurfural”, “hydroxymethylfurfural” and “HMF” may be used interchangeably in the context of the present invention. The IUPAC term of HMF is 5-(hydroxymethyl)-2-furaldehyde and it may also be used in the present context.

The term “enzymatic reaction” refers in the context of the present invention to a chemical reaction catalyzed by an enzyme, where “chemical reaction” refers to the general understanding of this term as a process of transforming one or more chemical substances into one or more other chemical substances.

The term “glucose isomerase” refers in the context of the present invention to an enzyme of E.C. 5.3.1.5 which is capable of catalysing the transformation of D-xylose to D-xylulose. Such enzymes are generally used in the high-corn syrup industry to convert glucose into fructose. In the context of the present invention glucose isomerase may be abbreviated to “GI” which is intended to encompass any glucose isomerase, e.g. independent of whether it is immobilized or not. As the currently available glucose isomerases are typically immobilized the term “IGI” may also be used which in the context of the present invention is intended to mean “immobilized glucose isomerase”.

The term “mannose isomerase” refers in the context of the present invention to an enzyme of E.C. 5.3.1.7 which is capable of catalysing the transformation of D-mannose to D-fructose,

The term “saccharide” refers in the context of the present invention to its well known meaning as an organic compound with the general formula Cₘ(H₂O)ₙ also known as a carbohydrate. Thus the term “saccharide” includes monosaccharides, disaccharides, oligosaccharides and polysaccharides.

The term “HFCS” refers in the context of the present invention to High Fructose Corn Syrup.

### DETAILED DESCRIPTION OF THE INVENTION

### Methods of the Present Invention

The first aspect of the invention relates to methods of producing 5-hydroxymethylfurfural (HMF) by dehydration of fructose and/or glucose, or alternatively fructose and/or mannose, comprising:
a) providing an aqueous solution comprising fructose and, optionally, glucose and/or mannose;
b) optionally contacting the solution with glucose isomerase enzyme (E.C. 5.3.1.5) which converts glucose to fructose and/or mannose isomerase enzyme (E.C. 5.3.1.7) which converts mannose to fructose;
c) combining the solution with at least one organic solvent as well as an acid catalyst and/or a salt to provide a reaction mixture, wherein the mixture forms a single-phase system at standard conditions of 20° C. and 1 atm. absolute pressure; and
d) heating said reaction mixture for a time sufficient to allow dehydration of fructose to provide 5-hydroxymethylfurfural in a resulting product mixture.

In a preferred embodiment, the aqueous solution in step (a) comprises glucose and/or mannose and step (b) is performed; preferably, the aqueous solution in step (a) contains at least 20 w/w % glucose and fructose, such as, a total of 30-90 w/w % fructose and glucose, e.g. 40-90 w/w % fructose and glucose, or a total of 50-90 w/w % fructose and glucose, or a total of 60-90 w/w % fructose and glucose; or preferably, the aqueous solution in step (a) contains least 20 w/w % mannose and fructose, such as, a total of 30-90 w/w % fructose and mannose, e.g. 40-90 w/w % fructose and mannose, or a total of 50-90 w/w % fructose and mannose, or a total of 60-90 w/w % fructose and mannose.

In another preferred embodiment of the first aspect, the glucose isomerase enzyme and/or the mannose isomerase enzyme is/are immobilized. Several immobilized isomerase enzymes are commercially available.

It is preferred, that the solution in step (c) comprises a concentration of carbohydrates above the solubilization limit.

In another preferred embodiment, the salt is a metal halide, such as NaCl, MgCl₂, LiCI, KCl, CaCl₂, CsCl, LiBr, NaBr, KBr or KI; preferably the salt is NaCl, as exemplified herein.

It is also preferred, that the concentration of the salt is in the range of 0.001-30% (w/w), preferably in the range of 0.01-20%(w/w), more preferably in the range of 0.1-10%(w/w), even more preferably in the range of 1-9%(w) and most preferably in the range of 2-8%(w/w).

In a preferred embodiment of the first aspect, the organic solvent is acetone, acetonitrile, dioxan, ethanol, methanol, n-propanol, isopropanol or tetrahydrofuran; preferably the organic solvent is acetone, as exemplified herein.

It may be advantageous to supply an acid catalyst to the reaction mixture and it is preferably a strong acid, such as HCl, HNO₃, H₂SO₄, H₃PO₄, or a weak acid, such as boric acid; preferably the acid catalyst is HCl, as exemplified herein. In some embodiments, the acid catalyst is combined with the organic solvent prior to combining the aqueous solution and the at least one organic solvent in step (c).

Another preferred embodiment relates to the pH value of the reaction mixture, which is preferably in the range of 1.0 to 10, such as in the range of pH 1.5-10, or in the range of pH 1.6-10, or in the range of pH 1.7-10, or in the range of pH 1.8-10, or in the range of pH 1.9-10, or in the range of pH 2.0-10, or in the range of 2.1-10, or in the range of pH 2.2-10, or in the range of pH 2.3-10, or in the range of pH 2.4-10, or in the range of pH 2.5-10, or in the range of pH 2.6-10, or in the range of pH 2.7-10, or in the range of pH 2.8-10, or in the range of pH 2.9-10, or in the range of pH 3 to 10, or in the range of pH 3 to 9, or in the range of pH 3.5 to 9, or in the range of pH 3 to 8, or in the range of pH 3.5 to 8, or in the range of 4 to 9, or in the range of pH 4 to 8.5, or in the range of pH 4 to 8, or in the range of pH 4.5 to 10, or in the range of pH 4.5 to 9, or in the range of pH 4.5 to 8.5, or in the range of pH 4.5 to 8, or in the range of pH 5 to 10, or in the range of pH 5 to 9, or in the range of pH 5 to 8.5, or in the range of pH 5 to 8, or in the range of pH 5.5 to 10, or in the range of pH 5.5 to 9, or in the range of pH 5.5 to 8.5, or in the range of pH 5.5 to 8, or in the range of pH 6 to 10, or in the range of pH 6 to 9, or in the range of pH 6 to 8.5, or in the range of pH 6 to 8.

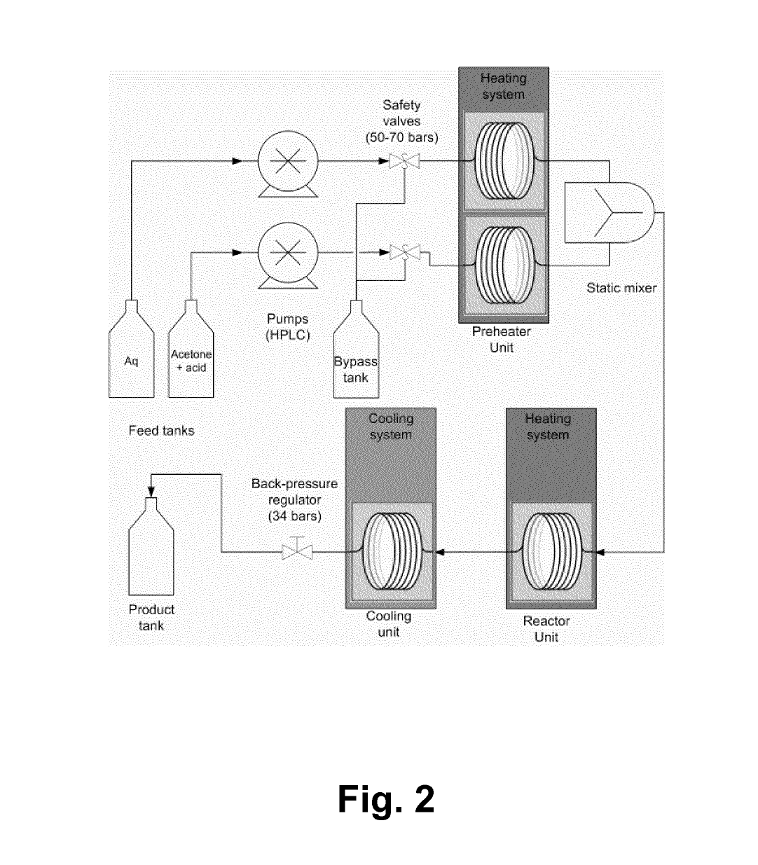

The aqueous solution and organic solvent may optionally be individually preheated prior to combining in step (c) (see FIG. 2). The preheated solutions may be combined to provide the reaction mixture of step (c) which is then heated in step (d) for a time sufficient to allow dehydration of fructose to provide 5-hydroxymethylfurfural. If preheating is used, the acid catalyst is preferably combined with the organic solvent prior to combining the aqueous solution and the at least one organic solvent in step (c).

Naturally, a process such as the HMF-production process of the first aspect may advantageously carried out continuously; accordingly, in a preferred embodiment of the first aspect, one or more of the steps are performed continuously.

Preferably, one or more steps in the process of the first aspect is carried out in a continuous flow reactor.

It is well-known that dehydration of fructose to HMF can lead to formation of polymers (e.g. humins), that may easily clog the pipes and vessels, which leads to difficulties, in particular where continuous processes are concerned. However, the present inventors have found that the use of an organic solvent in a single-phase aqueous reaction mixture keeps this at a manageable level. When combining this insight with the use of reactor vessels and tubes or pipes, where the insides have been at least partially lined with a non-stick material with similar properties to TEFLON®(DuPont).

Accordingly, in a preferred embodiment of the first aspect, one or more steps is carried out in a reactor or vessel the inside of which is at least partially lined or coated with a non-stick material, such as, polytetrafluoroethylene (PTFE), perfluoroalkoxy or fluorinated ethylene propylene. Further, it is preferred that the solution, reaction mixture or product mixture is transported between one or more vessels or process steps in tubes or pipes the inside of which is at least partially lined or coated with a non-stick material, such as, polytetrafluoroethylene (PTFE), perfluoroalkoxy or fluorinated ethylene propylene.

Preferably, in the method of the first aspect, the at least one organic solvent is recovered from the product mixture and recycled to step (c) of the process; preferably the at least one organic solvent is recovered by distillation from the product mixture and recycled to step (c) of the process.

It is also preferable in a method of the first aspect, that the 5-hydroxymethylfurfural is recovered from the product mixture, and wherein any remaining reaction mixture still comprising unreacted fructose and/or glucose and/or mannose is then combined with glucose isomerase enzyme (E.C. 5.3.1.5) which converts glucose to fructose and/or mannose isomerase enzyme (E.C. 5.3.1.7) which converts mannose to fructose, and the resulting medium is then recycled to step (c) of the process. In some embodiments, reaction byproducts, such as humins, are partially or fully removed before recycling unreacted fructose, glucose and/or mannose.

### Use of HMF

The HMF produced by any of the above mentioned first and second methods may be further processed to obtain another product. Examples of such products include but are not limited to 2,5-furandicarboxylic acid (FDCA), diformylfuran (DFF), formylfuran carboxylic acid (FFCA), 2,5-dimethylfuran (DMF), and p-xylene.

The HMF produced by any of the above mentioned processes may in particular be oxidized to produce 2,5-furandicarboxylic acid, diformylfuran (DFF) or formylfuran carboxylic acid (FFCA). Hence any of the above mentioned methods may comprise a further step of oxidizing the obtained HMF to 2,5-furandicarboxylic acid.

Examples of methods suitable for oxidizing HMF to 2,5-furandicarboxylic acid include but are not limited to those described in US patents U.S. Pat. No. 4,977,283 and U.S. Pat. No. 7,411,078, and US patent application US 2008/0103318.

U.S. Pat. No. 4,977,283 describes a process for the oxidation of 5-hydroxymethylfurfural which comprises oxidizing 5-hydroxymethylfurfural in an aqueous medium with oxygen in the presence of a catalyst which contains at least one metal of the platinum group.

U.S. Pat. No. 7,411,078 describes oxidizing e.g. 5-hydroxymethylfurfural with a metal permanganate in an alkaline environment to produce 2,5-furandicarboxylic acid. Advantageously, the alkaline environment contains at least one of alkali metal hydroxides and alkali earth metal hydroxides, and the oxidation is performed at a temperature of from 1 to 50° C.

US 2008/01003318 describes a method of oxidizing hydroxymethylfurfural (HMF) includes providing a starting material which includes HMF in a solvent comprising water into a reactor. At least one of air and O₂ is provided into the reactor. The starting material is contacted with a catalyst comprising Pt on a support material where the contacting is conducted at a reactor temperature of from about 50° C. to about 200° C.
Hence any of the methods of the present invention may comprise as a further step a process of oxidizing HMF to 2,5-furandicarboxylic as described above.

Furthermore, the present invention also relates to the products obtained by any method according to the present invention.

### Compositions

The present invention relates to the production of hydroxymethylfurfural by dehydration of fructose and/or glucose.

The methods of the present invention may use different starting materials, i.e. a composition comprising fructose, a composition comprising glucose, a composition comprising mannose, a composition comprising glucose and fructose, a composition comprising glucose and mannose, a composition comprising fructose and mannose or a composition comprising fructose, glucose and mannose. As these compositions may have certain features in common the term “starting material” used in the following refers to all the listed compositions. Often such industrially produced compositions comprise different saccharides, such as both glucose and fructose, or both fructose and mannose or even all three of fructose, glucose and mannose, however the present invention is not limited to such composition as compositions which have been purified with respect to either glucose, mannose or fructose can also be used.

The term “composition” is in the context of the present invention to be understood in its broadest context; however it may typically be an aqueous solution.

The compositions used in the present invention as starting materials may typically contain a total of at least 20% (w/w) glucose and/or mannose and/or fructose.

The starting material preferably contains at least 20 w/w % glucose and fructose, such as, a total of 30-90 w/w % fructose and glucose; e.g. 40-90 w/w % fructose and glucose, or a total of 50-90 w/w % fructose and glucose, or a total of 60-90 w/w % fructose and glucose.

The starting material preferably contains least 20 w/w % mannose and fructose, such as, a total of 30-90 w/w % fructose and mannose, e.g. 40-90 w/w % fructose and mannose, or a total of 50-90 w/w % fructose and mannose, or a total of 60-90 w/w % fructose and mannose.

As the compositions used as starting materials in the methods of the present invention in many cases may be obtained from natural sources, e.g. biomass, they may also contain other components than fructose and/or glucose and/or mannose including other saccharides. For example the compositions used as starting material in the methods of the present invention may comprise 0-10 w/w % oligosaccharides.

The choice of starting material may to some extent affect the combination of steps in a method of the present invention. Furthermore, the starting compositions used in the methods or processes of the present invention may as described above comprise other saccharides than fructose, glucose and mannose.

For example if a composition comprises a relative high amount of fructose it may be used directly as a starting material for the dehydration process of fructose to HMF. In this context a “relative high amount of fructose” may typically be a composition wherein at least 40 w/w % of the total amount of saccharides in the composition is fructose or that fructose constitutes at least 40 w/w % of the total amount of saccharides in the composition.

Thus the compositions used in the present invention, i.e. a composition comprising fructose, a composition comprising fructose and mannose, and a composition comprising fructose and glucose, and a composition comprising fructose, glucose and mannose may in a particular embodiment be a composition wherein 40-100 w/w % of the total amount of saccharides in the composition is fructose. More particularly 45-100 w/w % of the total amount of saccharides may be fructose, or 45-95 w/w % of the total amount of saccharides may be fructose, or 50-95 w/w % of the total amount of saccharides may be fructose.

Examples of compositions wherein fructose constitutes more than 40 w/w % of the total amount of saccharides present in the composition include but are not limited to HFCS (high fructose corn syrup), invert sugar, inulin and compositions which have been purified with respect to fructose.

HFCS typically comprise 40-60 w/w % fructose of the total amount of saccharides. Moreover, the ratio of fructose to glucose in HFCS is typically between 40:60 and 60:40, such as a ratio between 44:56 and 46:54, more particularly a ratio of 45:55. In some cases the ratio of fructose to glucose in HFCS may be in the range of 53:47 to 59:41, or in the range of 40:60 to 44:56.

Invert sugar also known as inverted sugar syrup, arise from hydrolysis of sucrose and invert sugar therefore typically comprises fructose and glucose in a ratio of approximately between 48:52 and 52:48, such as a ratio between 49:51 and 51:49, more particularly a ratio of 50:50. Thus fructose typically constitute 48-52 w/w % of the total amount of saccharides in invert sugar, in particular 49-51 w/w % of the total amount of saccharides is fructose, even more particularly 50 w/w % of the total amount of saccharides is fructose. Glucose similarly constitute 48-52 w/w % of the total amount of saccharides in invert sugar, in particular 49-51 w/w % of the total amount of saccharides in invert sugar is glucose, even more particularly 50 w/w % of the total amount of saccharides in invert sugar is glucose.

Inulins are polymers that mainly comprises fructose units joined by a β(2→1) glycosidic bond and which typically have a terminal glucose units. Hydrolysis of inulin typically results in a composition wherein approximately 90 w/w %, e.g. in the range of 85-95 w/w %, of the total amount of saccharides is fructose and approximately 10 w/w %, e.g. in the range of 5-15 w/w %, of the total amount of saccharides is glucose.

If on the other hand a composition comprising a relative high concentration of glucose or mannose, and a relative low concentration of fructose is used as a starting material in a method of the present invention it is an advantage to include a step of increasing the amount of fructose relative to the amount of glucose or mannose, prior to using it in the dehydration step of the present invention. Methods of increasing the amount of fructose in a composition are described above but it may also involve other methods such as purification of fructose. In this context a “relative high concentration of glucose or mannose” means a composition wherein 60-100 w/w % of the total amount of saccharides is glucose or mannose, such as 60-95 w/w % of the total amount of saccharides is glucose or mannose.

Furthermore, in this context the term “relative low concentration of fructose” means a composition wherein fructose constitutes 40 w/w % or less than 40 w/w % of the total amount of saccharides, i.e. wherein 0-40 w/w % of the total amount of saccharides is fructose.

Examples of such compositions comprising a high concentration of glucose and a low concentration of fructose include but are not limited to glucose obtained from any source of starch, such as but not limited to corn, wheat and potatoes, glucose obtained from cellulosic biomass, e.g. fibres, stovers, wheat, or straw. The glucose may also be obtained from other sources of starch or biomass known to a person skilled in the art.

Glucose obtained from starch typically, results in a composition wherein approximately 92-98 w/w % of the total amount of saccharides is glucose.

Converting glucose to fructose by an enzymatic reaction catalyzed by glucose isomerase typically results in a composition wherein approximately 43-47 w/w % of the total amount of saccharides is fructose and approximately 53-57 w/w % of the total amount of saccharides is glucose. Thus the ratio of fructose to glucose in these compositions may typically be in range of 43:57 and 47:53, such as in the range of 44:56 and 46:54, or approximately 45:55.

Examples of compositions comprising a high concentration of mannose and a low concentration of fructose include but are not limited to palm kernel cake.

Mannose may in a particular embodiment be converted to fructose by an enzymatic reaction catalyzed by mannose isomerase.

### Reaction Mixture

The processes of converting fructose or glucose or mannose to HMF take place in a reaction mixture that is a mixture of an aqueous solution and one or more organic solvents that are fully miscible with water, the mixture forming a one phase system at standard conditions of 20° C. and 1 atm. absolute pressure. Thus the reaction mixture of the present invention comprises a single phase system which typically may be liquid due to the nature of the components involved and the dehydration process. In the context of the present invention the term “phase” refers to the solubility of the aqueous solution in the one or more organic solvent and vice versa. Thus in the context of the present invention it means that the solubility of the aqueous solution in the organic solvent and vice versa is so high that the reaction mixture comprises only one single distinct phase; i.e. a mixture of the aqueous solution and the one or more organic solvents.

The reaction mixture of the present invention may comprise more than or less than 50 v/v % organic solvent. Hence the amount of other solvents than water in the reaction mixture may in particular be in the range of 50-100 v/v % organic solvent or 0-50 v/v % organic solvent. In some embodiments, the reaction mixture comprises greater than 50 v/v % organic solvent, such as greater than 60 v/v %, 65 v/v %, 70 v/v %, 75 v/v %, 80 v/v %, 85 v/v %, 90 v/v %, or 95 v/v % organic solvent. In some embodiments, the reaction mixture may be in the range of 50-100 v/v %, such as 55-90 v/v %, 60-80 v/v %, or 65-70 v/v % organic solvent. In other embodiments, the reaction mixture comprises less than 50 v/v % organic solvent, such as less than 45 v/v %, 40 v/v %, 35 v/v %, 30 v/v %, 25 v/v %, 20 v/v %, 15 v/v %, 10 v/v %, or 5 v/v % organic solvent.

As described herein, the inventors of the present invention have surprisingly found that the presence of salt in the reaction mixture is capable of catalyzing dehydration of fructose to HMF. In the context of the present invention the term “salt” is to be understood as an ionic compound composed of cations (positively charged ions) and anions (negative ions) so that the product is electrically neutral (without a net charge). These component ions can be inorganic such as Chloride (Cl⁻), as well as organic such as acetate (CH₃COO⁻) and monoatomic ions such as fluoride (F⁻), as well as polyatomic ions such as sulfate (SO₄²⁻), or monovalent ions, such as Na⁺, or divalent ions, such as Mg²⁺. There are several varieties of salts. Salts that produce hydroxide ions when dissolved in water are basic salts and salts that produce hydronium ions in water are acid salts. Neutral salts are those that are neither acid nor basic salts. Zwitterions contain an anionic center and a cationic center in the same molecule but are not considered to be salts. Examples include amino acids, many metabolites, peptides and proteins. When salts are dissolved in water, they are called electrolytes, and are able to conduct electricity, a property that is shared with molten salts.

The salt present in the aqueous phase may in particular be an inorganic salt, such as a salt selected from the group consisting of but not limited to metal halides, metal sulphates, metal sulphides, metal phosphates, metal nitrates, metal acetates, metal sulphites and metal carbonates. Examples of such salts include but are not limited to sodium chloride (NaCl), sodium sulphite (Na₂SO₃), magnesium chloride (MgCl₂), lithium chloride (LiCI), potassium chloride (KCl), calcium chloride (CaCl₂), cesium chloride (CsCl), sodium sulphate (Na₂SO₄), potassium sulphate (K₂SO₄), lithium bromide (LiBr), sodium bromide (NaBr), potassium bromide (KBr), lithium nitrate (LiNO₃), sodium nitrate (NaNO₃), potassium nitrate (KNO₃) and potassium iodine (KI). The salt may in particular be a metal halide, such as NaCl, MgCl₂, LiCI, KCl, CaCl₂, CsCl, LiBr, NaBr, KBr or KI.

The concentration of salt may depend on the choice of salt, however it may for many or most salts be in the range of 0.1-30 w/w %, such as in the range of 0.5-30 w/w %, or in the range of 1-30 w/w %, or in the range of 0.1-25 w/w %, or in the range of 0.5-25 w/w %, or in the range of 1-25 w/w %, or in the range of 0.1-20 w/w %, or in the range of 0.5-20 w/w %, or in the range of 1-20 w/w %, or in the range of 0.5-15 w/w %, or in the range of 0.5-10 w/w %, or in the range of 0.5-7.5 w/w %, or in the range of 1-10 w/w %, or in the range of 1-7.5 w/w %, or in the range of 1-5 w/w %, or in the range of 2-10 w/w %, or in the range of 2-7.5 w/w %, or in the range of 2-5 w/w %.

The inventors of the present invention have shown that by combining the salt with a weak acid, such as boric acid, the HMF yield and fructose conversion is increased even further.

Without being bound by any theory, the inventors of the present invention are of the opinion that the combination of the sugars (e.g. fructose or glucose) and salt may affect the acidic effect of the weak acid causing it to behave more acidic than without the presence of sugar and salt. Hence, in a particular embodiment the aqueous phase may comprise a weak acid.

In the context of the present invention a weak acid is an acid with a pKₐ-value which is 1 or higher than 1 (pKₐ(weak acid)≧1). Examples of such acids include boric acid (B(OH)₃). The amount of weak acid, e.g. boric acid, in the aqueous phase may typically be in the range of 0.1-200 g/L, such as in the range of 5-200 g/L, or in the range of, 10-200 g/L, or in the range of 10-150 g/L, or in the range of 25-150 g/L, or in the range of 50-150 g/L, or in the range of 50-125 g/L, or in the range of 75-125 g/L, such as 100 g/L.

Addition of a weak acid such as boric acid to the reaction mixture does not decrease the pH as much as when using a strong acid as a catalyst. Thus the advantages of using salt as catalyst compared to using a strong acid also applies to using a combination of salt and a weak acid, such as boric acid, or a strong acid, such as hydrochloric acid, as a catalyst.

For the process of dehydrating fructose to HMF, the reaction mixture may in a particular embodiment have a pH in the range of pH 1.0 to 10, such as in the range of pH 1.5-10, or in the range of pH 1.6-10, or in the range of pH 1.7-10, or in the range of pH 1.8-10, or in the range of pH 1.9-10, or in the range of pH 2.0-10, or in the range of 2.1-10, or in the range of pH 2.2-10, or in the range of pH 2.3-10, or in the range of pH 2.4-10, or in the range of pH 2.5-10, or in the range of pH 2.6-10, or in the range of pH 2.7-10, or in the range of pH 2.8-10, or in the range of pH 2.9-10, or in the range of pH 3 to 10, or in the range of pH 3 to 9, or in the range of pH 3.5 to 9, or in the range of pH 3 to 8, or in the range of pH 3.5 to 8, or in the range of 4 to 9, or in the range of pH 4 to 8.5, or in the range of pH 4 to 8, or in the range of pH 4.5 to 10, or in the range of pH 4.5 to 9, or in the range of pH 4.5 to 8.5, or in the range of pH 4.5 to 8, or in the range of pH 5 to 10, or in the range of pH 5 to 9, or in the range of pH 5 to 8.5, or in the range of pH 5 to 8, or in the range of pH 5.5 to 10, or in the range of pH 5.5 to 9, or in the range of pH 5.5 to 8.5, or in the range of pH 5.5 to 8, or in the range of pH 6 to 10, or in the range of pH 6 to 9, or in the range of pH 6 to 8.5, or in the range of pH 6 to 8.

For the process of dehydrating glucose to HMF, the pH of the reaction mixture may in particular be in the range of 1 to 9, such as a pH in the range of 1 to 8, or in the range of 1 to 7, or in the range of 1 to 6, or in the range of 1 to 5, or in the range of 1 to 4, or in the range of 1.5 to 8, or in the range of 1.5 to 7, or in the range of 1.5 to 6, or in the range of 1.5 to 5, or in the range of 1.5 to 4.

The dehydration of glucose and/or fructose and/or mannose to HMF takes place in the reaction mixture and the process may create by-products. Some of these by-products are acidic and they may therefore cause the pH of the aqueous phase to fall, as the dehydration of glucose and/or fructose and/or mannose to HMF takes place. Thus in the context of the present invention the pH range of the reaction mixture refers to t₀ of the dehydration process. In other words, it is the pH of the reaction mixture at that point in time, where all components are present, but prior to any actual dehydration of fructose or glucose or mannose to HMF.

For example, if the method of the present invention is run as a continuous process on an industrial scale, the pH of a composition comprising fructose, glucose, mannose, fructose and glucose, fructose and mannose, mannose and glucose, or all three fructose, glucose and mannose may be the same as the pH of the reaction mixture at tₒ, when no acidic catalysts are added to the reaction mixture.

For example, if the starting material, i.e. the composition comprising fructose, fructose and mannose, or fructose and glucose, used for the dehydration of fructose to HMF, has been obtained from conversion of glucose to fructose, or mannose to fructose, by an enzymatic reaction catalyzed by a glucose isomerase or mannose isomerase, the pH of the composition obtained from this conversion will typically be in the range of 6.5-7.5. As glucose isomerase currently is used on an industrial basis in the form of columns to which the glucose isomerase is immobilized, this means that the pH of the composition leaving the glucose isomerase may typically be in the range of 6.5-7.5. It may of course be possible to adjust the pH of this composition before it enters the dehydration process.

In alternative embodiment, the reaction mixture for the process of dehydrating fructose to HMF does not contain an acidic catalyst or does not comprise a strong acid. In the context of the present invention “does not contain an acidic catalyst” means that no acidic catalyst has been added to the reaction mixture. An “acidic catalyst” may in particular be an acid which has a pKₐ-value below 5, such as a pKₐ-value below 4, or a pKₐ-value below 3, or a pKₐ-value below 2, or have a pKₐ-value between 1-5, such as between 1-4, or between 1-3 or between 1-2, or between 1-1.5, or between 2-4, such as between 2-3, or between 2.5-3.5; or between 1.5-4, such as between 1.5-3, or between 1.5-2.5; or between 3-5, such as between 3.5-4.5 or between 3-4, or between 4-5. An “acidic catalyst” may in particular be a “strong acid”, wherein a strong acid is an acid with a pKₐ-value below 1. A “strong acid” in the context of the present invention is to be understood as an acid with a pKₐ-value which is lower than 1 (pKₐ(strong acid)<1). Examples of such acidic catalysts include but are not limited to mineral acids, such as HCl, HNO₃, H₂SO₄, H₃PO₄, sulfonic sulfonic acid resins, zeolites, acid-functionalized Mobil composition materials (MCM's), sulphated zirconia, heteropolyacids, phosphates such as NbOPO₄, vanadium phosphate, solid silica- and silica-alumina, Brøndsted or Lewis acid catalyst.

The inventors of the present invention has surprisingly found out that the salt present in the reaction mixture is able to function as catalyst for the dehydration of fructose to HMF, making it unnecessary to use other catalysts such as acidic catalysts which have previously been used.

Hence, in a particular embodiment the reaction mixture of the present invention does not comprise an acidic catalyst or does not comprise a strong acid. Although the inventors of the present invention found out that it is not necessary to use an acidic catalyst for the dehydration of fructose to HMF, such catalysts may still be present in the reaction mixture, for example, in small amounts. Thus any of the above mentioned catalysts may be present in the reaction mixture.

Furthermore, for the process of dehydration of glucose to HMF, or mannose to HMF, it may also be an advantage to include an acidic catalyst, such as AlCl₃ to minimize the production of unwanted side-products. The optimal reaction conditions for the dehydration of fructose, mannose and glucose, respectively, to HMF are not the same.

The reaction mixture also comprises an organic solvent. A suitable organic solvent is a solvent which is miscible with the aqueous solution of the reaction mixture at standard conditions of 20° C. or higher and 1 atm. absolute pressure. Examples of such organic solvents include in particular but are not limited to alcohols, ketones, or combinations thereof.

In a particular embodiment the organic solvent may be acetone. Other examples of useful organic solvents include but are not limited to low-molecular weight alcohols (e.g., fusel oil, isoamyl alcohol, butanol or isopentyl alcohol, straight or branched alcohols, such as pentanol, tertbutyl alcohol or 1-butanol, straight or branched alkanones, such as butanone, pentanone, hexanone, heptanone, diisobutylketone).

### Examples

### Flow Reactor for Dehydration of Sugars

Dehydrations were carried out in a continuous flow reactor setup, where organic solvents and aqueous solutions of sugars and catalyst were separately pumped through a tube reactor using HPLC pumps with pressure indicators (Smartline 100, Knauer, Berlin, Germany). The reactor tubes consisted of stainless steel tubing coil (outer diameter (OD): ⅛″; inner diameter (ID): 0.07″), of which some were in-lined with PTFE tubing (OD. 1/16″, ID. 1 mm). The coiled reactors were submerged in an oil bath, which was heated and stirred on a magnetic stirrer/heating plate with temperature control (RCT basic, IKA, Staufen, Germany). The outlet tubing was connected to an in-line filter, consisting of a stainless steel column filled with cotton and submerged in a water bath for fast cooling of the reaction mixture. The outlet of the filter was connected to a fixed pressure regulator (IDEX, Washington, U.S.A.), for maintaining a fixed pressure in the reactor tube. Collected samples were filtered through a syringe filter and analyzed by HPLC on an Aminex HPX-87H (Biorad, Hercules, Calif.) column at 60° C. with 0.6 mL/min 0.005 M aqueous sulphuric acid as eluent. Compounds were quantified using a refractive index detector by external calibration with authentic compounds.

The results presented in examples are calculated in the following way:

Yield of HMF:

$${Y}_{i} = \frac{{C}_{HMF}}{{C}_{0 , Fructose}}$$

Conversion of fructose:

$${X}_{f} = \frac{{C}_{0 , Fructose} - {C}_{Fructose}}{{C}_{0 , Fructose}}$$

Selectivity of HMF from fructose:

$${S}_{HMF , fruc} = \frac{{C}_{HMF}}{{C}_{0 , Fructose} - {C}_{Fructose}}$$

The residence time:

$$τ = \frac{Volume}{volumetric flow}$$

### Example 1

### Impact of Solvent on the Pressure Build-Up in the Reactor System

Fructose and glucose/fructose mixtures were dehydrated in the above-mentioned flow reactor system, using both acetone and MIBK as solvent. It was found that when acetone was used as solvent and the reactor coil was in-lined with PTFE tubing, only little pressure increase was observed (1-7 bar). When MIBK was used as solvent and/or when the reactor tubes were not in-lined with PTFE tubing, then a significant increase in pressure over time was observed, due to clogging of the reactor system with insoluble polymeric materials.

### Example 2

### Dehydration of Fructose Using Acetone as Solvent

Aqueous solutions of fructose, with hydrochloric acid and/or sodium chloride as dehydration catalyst were dehydrated in the above reactor using acetone as organic solvent. The reaction conditions and results are found in Table 1. The results show, that fructose is converted to HMF with high selectivity at high conversions using both NaCl/HCl (Table 1, entry 1-2), HCl (Table 1, entry 3-7) and NaCl (Table 1, entry 8) as catalyst. The reaction rate was found to significantly increase in the presence of sodium chloride, as indicated by twice as fast reaction rate when using sodium chloride in combination with hydrochloric acid (Table 1, entry 2 vs. entry 7).

TABLE 1
Results for the dehydration of fructose in the presence of acetone

| | Reaction | | Reaction | Reaction | | | |
|---|---|---|---|---|---|---|---|
| | Conditions | Reaction | mixture | mixture | Conversion | HMF | HMF |
| Entry | (Temp/τ) | mixture [cat.] | solvent | [sugar] | of sugars | yield | selectivity |
| 1 | 210° C./0.23 min | 16.7 g/L NaCl; | Acetone:H₂O | 99 g/L | 87% | 66% | 75% |
| | | 3.3 mM HCl | 2:1 | fructose | | | |
| 2 | 210° C./0.23 min | 16.7 g/L NaCl; | Acetone:H₂O | 99 g/L | 90% | 65% | 73% |
| | | 3.3 mM HCl | 2:1 | fructose | | | |
| 3 | 210° C./0.25 min | 16.7 g/L NaCl; | Acetone:H₂O | 99 g/L | 90% | 65% | 73% |
| | | 3.3 mM HCl | 2:1 | fructose | | | |
| 4 | 210° C./0.25 min | 3.3 mM HCl | Acetone:H₂O | 99 g/L | 69% | 49% | 71% |
| | | | 2:1 | fructose | | | |
| 5 | 210° C./0.30 min | 3.3 mM HCl | Acetone:H₂O | 99 g/L | 78% | 55% | 70% |
| | | | 2:1 | fructose | | | |
| 6 | 210° C./0.37 min | 3.3 mM HCl | Acetone:H₂O | 99 g/L | 85% | 60% | 71% |
| | | | 2:1 | fructose | | | |
| 7 | 210° C./0.46 min | 3.3 mM HCl | Acetone:H₂O | 99 g/L | 90% | 64% | 71% |
| | | | 2:1 | fructose | | | |
| 8 | 200° C./3.04 min | 16.7 g/L NaCl | Acetone:H₂O | 99 g/L | 79% | 57% | 72% |
| | | | 2:1 | fructose | | | |

### Example 3

### Selective Dehydration of Fructose/Glucose Mixture Using Acetone as Solvent

An aqueous solution of 128 g/L fructose, 172 g/L glucose, 50 g/L sodium chloride, and 0.01 M hydrochloric acid was dehydrated in the above flow reactor using two volumes of acetone as solvent at different temperatures and residence times. Results are shown in Table 2.

TABLE 2
Results of dehydration of HFCS in the presence of acetone

| | Reaction | | | Reaction | | | |
|---|---|---|---|---|---|---|---|
| | Conditions | Reaction mixture | | mixture | Conversion | HMF | HMF |
| Entry | (Temp/τ) | [cat.] | Solvent | [sugar] | of fructose | yield | selectivity |
| 1 | 200° C./0.66 min | 3.3 mM HCl | Acetone:H₂O | 100 g/L | 90% | 62% | 69% |
| | | | 2:1 | | | | |
| 2 | 200° C./0.99 min | 3.3 mM HCl | Acetone:H₂O | 100 g/L | 97% | 69% | 72% |
| | | | 2:1 | | | | |
| 3 | 200° C./0.48 min | 3.3 mM HCl | Acetone:H₂O | 100 g/L | 93% | 61% | 66% |
| | | | 2:1 | | | | |
| 4 | 200° C./0.52 min | 16.7 g/L NaCl; | Acetone:H₂O | 100 g/L | 91% | 68% | 75% |
| | | 3.3 mM HCl | 2:1 | | | | |

### Example 4

### Introduction of Preheater

A preheater may be introduced to preheat the substrate mixture and solvent separately. In one experiment, the substrate mixture consisted of 128 g/L fructose and 172 g/L glucose, and the acetone solvent was separately mixed with 10 mM HCl. The preheater (HC stainless steel O.D. ⅛″ I.D. 0.07″ with in-lined Teflon tubing O.D 1/16″ I.D. 0.1 mm) was then introduced after the bypass security valves and before the reactor as shown in FIG. 2.

The preheater reached temperatures of 170-190° C. for both lines before entry into a mixer at a volumetric ratio of 2:1 for solvent:substrate. The mixture was hereafter led to the reactor wherein dehydration occurred at 180-200° C. Results are shown in Table 3.

TABLE 3
Results of dehydration of HFCS in the presence of acetone using a preheater

| | Reaction | | | Reaction | | | |
|---|---|---|---|---|---|---|---|
| | Conditions | Reaction mixture | | mixture | Conversion | HMF | HMF |
| Entry | (Temp/τ) | [cat.] | Solvent | [sugar] | of fructose | yield | selectivity |
| 1 | 190/180° C. | 16.7 g/L NaCl; | Acetone:H₂O | 100 g/L | 82% | 62% | 75% |
| | 0.25 min | 3.3 mM HCl | 2:1 | | | | |
| 2 | 190/180° C. | 16.7 g/L NaCl; | Acetone:H₂O | 100 g/L | 93% | 71% | 76% |
| | 0.50 min | 3.3 mM HCl | 2:1 | | | | |
| 3 | 190/200° C. | 16.7 g/L NaCl; | Acetone:H₂O | 100 g/L | 94% | 71% | 74% |
| | 0.13 min | 3.3 mM HCl | 2:1 | | | | |

1. A process for producing 5-hydroxymethylfurfural, said process comprising:
a) providing an aqueous solution comprising fructose and, optionally, glucose and/or mannose;
b) optionally contacting the solution with glucose isomerase enzyme (E.C. 5.3.1.5) which converts glucose to fructose and/or mannose isomerase enzyme (E.C. 5.3.1.7) which converts mannose to fructose;
c) combining the solution with at least one organic solvent as well as an acid catalyst and/or a salt to provide a reaction mixture, wherein the mixture forms a single-phase system at standard conditions of 20° C. and 1 atm. absolute pressure; and
d) heating said reaction mixture for a time sufficient to allow dehydration of fructose to provide 5-hydroxymethylfurfural in a resulting product mixture.

2. The process of claim 1, wherein the aqueous solution in step (a) comprises glucose and/or mannose and step (b) is performed.

3. The process of claim 2, wherein the aqueous solution in step (a) contains at least 20 w/w % glucose and fructose.

4. The process of claim 2, wherein the aqueous solution in step (a) contains least 20 w/w % mannose and fructose.

5. The process of claim 1, wherein the glucose isomerase enzyme and/or the mannose isomerase enzyme is/are immobilized.

6. The process of claim 1, wherein the solution in step (c) comprises a concentration of carbohydrates above the solubilization limit.

7. The process of claim 1, wherein the salt is a metal halide.

8. The process of claim 1, wherein the concentration of the salt is in the range of 0.001-30%(w/w).

9. The process of claim 1, wherein the organic solvent is acetone, acetonitrile, dioxan, ethanol, methanol, n-propanol, isopropanol or tetrahydrofuran.

10. The process of claim 1, wherein the acid catalyst is a strong acid.

11. The process of claim 1, wherein the reaction mixture has a pH in the range of 1.0 to 10.

12. The process of claim 1, wherein one or more of the steps are performed continuously.

13. The process of claim 1, wherein one or more steps is carried out in a continuous flow reactor.

14. The process of claim 1, wherein one or more steps is carried out in a reactor or vessel the inside of which is at least partially lined or coated with a non-stick material.

15. The process of claim 1, wherein the solution, reaction mixture or product mixture is transported between one or more vessels or process steps in tubes or pipes the inside of which is at least partially lined or coated with a non-stick material.

16. The process of claim 1, wherein the at least one organic solvent is recovered from the product mixture and recycled to step (c) of the process.

17. The process of claim 1, wherein the 5-hydroxymethylfurfural is recovered from the product mixture, and wherein any remaining reaction mixture still comprising unreacted fructose and/or glucose and/or mannose is then combined with glucose isomerase enzyme (E.C. 5.3.1.5) which converts glucose to fructose and/or mannose isomerase enzyme (E.C. 5.3.1.7) which converts mannose to fructose, and the resulting medium is then recycled to step (c) of the process.

18. The process of claim 17, wherein humins, if any, is partially or fully removed from the remaining reaction mixture prior to being combined with glucose isomerase enzyme and/or mannose isomerase enzyme.

19. The process of claim 1, wherein the aqueous solution and at least one organic solvent are preheated prior to combining in step (c).

20. The process of claim 1, wherein the acid catalyst is combined with the organic solvent prior to combining the aqueous solution and the at least one organic solvent in step (c).
